(19) United States
(12) Patent Application Publication
(10) Pub. No.: US 2006/0081260 A1
(43) Pub. Date: Apr. 20, 2006

(54) Percutaneous introducer balloon

(76) Inventors: Scott E. Eells, Bloomington, IN (US); Thomas A. Osborne, Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(21) Appl. No.: 11/086,622

(22) Filed: Mar. 22, 2005

Related U.S. Application Data
(60) Provisional application No. 60/555,581, filed on Mar. 23, 2004.

(51) Int. Cl. A62B 9/06; A61M 16/00

(52) U.S. Cl. 128/207.29; 128/207.14; 128/207.15

(57) ABSTRACT

An apparatus and method for use in the percutaneous placement of a medical device, such as a tracheostomy tube. An elongated hollow tube has an inflatable dilator balloon mounted thereon. The balloon comprises a distal portion, an intermediate portion and a proximal portion. The medical device is carried on the intermediate portion. At least a segment of the distal portion has an inflated outer diameter that is at least as large as the outer diameter of the medical device. The inflated outer diameter of the intermediate portion is sized relative to an internal diameter of the medical device to hold the medical device thereon. An inflation assembly is provided to enable the balloon to be selectively inflated and deflated. A body opening is dilated with the inflated dilator balloon, and the medical device may be percutaneously placed across the dilated opening. Following placement of the device, the dilator balloon may be deflated and withdrawn from the apparatus through a lumen of the medical device.

### RELATED APPLICATION

The present patent document claims the benefit of the filing date under 35 U.S.C. §119(e) of Provisional U.S. Patent Application Ser. No. 60/555,581, filed Mar. 23, 2004, which is hereby incorporated by reference.

### BACKGROUND

1. Technical Field

The present invention relates to an introducer apparatus. The invention also relates to a method for placing a medical interventional device, such as a tracheostomy tube, in a designated area of a patient. More specifically, the invention relates to an introducer balloon assembly for use in the percutaneous insertion of a tracheostomy tube into the trachea of a patient.

2. Background Information

Tracheostomy tubes are utilized to assist a patient's breathing when an obstruction is present in the patient's throat which hinders or prevents normal breathing. When the use of tracheostomy tubes was first introduced in the medical field, such tubes were generally inserted surgically through an incision in the trachea made by a physician below the obstruction. During this procedure, tracheal cartilage was generally severed and portions of the cartilage were removed to create a stoma of sufficient size to enable insertion of the tracheostomy tube.

Recent advances in the design and use of tracheostomy tubes have enabled physicians to replace this invasive surgical procedure with much less invasive percutaneous insertion procedures. Many such procedures have utilized the well-known Seldinger technique for placement of the tracheostomy tube. Numerous patents and other publications describe the design of the various tracheostomy tubes, as well as suitable methods for percutaneously placing such tubes. A general background of tracheostomy procedures is provided in U.S. Pat. No. 5,058,580. Representative patents that illustrate aspects of this procedure include U.S. Pat. Nos. 6,662,804 6,382,209, 5,217,005, 4,364,391, 4,889,112 and 6,286,509. All of the aforementioned patents are incorporated herein by reference.

Percutaneously-placed tracheostomy tubes are generally inserted through a small opening made in the trachea. Initially, a puncture is made in the trachea with a suitably-sized needle. A tapered dilator, or a series of tapered dilators of increasing diameter, is inserted through the puncture to increase the diameter of the opening such that the tracheostomy tube can be inserted over the dilator into the dilated opening. The dilator is then removed, leaving the tracheostomy tube in place.

Although the percutaneous placement of tubes is advantageous when compared to surgical tube placement, there continue to be disadvantages incurred during percutaneous placement. One disadvantage is that considerable longitudinal (axial) pushing force is required on the dilator(s) in order to dilate the needle puncture in a radial direction to a size sufficient to allow introduction of a tracheostomy tube. Such an axially-directed force can produce trauma to the tracheal entry site that may further complicate the patient's condition. In addition, this axially directed force on the trachea may result in the inadvertent impingement of the dilator tips and wire guides on the tracheal wall opposite the entry point, thereby unnecessarily injuring the trachea.

Another disadvantage is that the use of multiple, increasing diameter dilators requires that the axially-directed pushing force be applied multiple times. The result of such multiple pushing forces is that the combined trauma caused by such multiple pushing forces can exceed the trauma caused by a single, large diameter, tapered dilator. Yet another disadvantage is that the transition between the dilator/obturator and the distal end of the tracheostomy tube may create a “bump”, or ridged surface, at the transition site, which surface must ultimately be forced through the puncture site. Normally this bump, or ridged surface, is difficult to force through the tracheal cartilage. Although the physician can overdilate the puncture site in order to eliminate this transitional bump, overdilation is inherently undesirable because of the desire to maintain the dilated opening as small as possible so that an air-tight seal is formed around the tube. Air leaks around the tube caused by overdilation of the opening interfere with the use of the tube for assisted breathing.

It is desired to provide an introducer assembly for percutaneous placement of a medical interventional device, such as a tracheostomy tube, that overcomes the problems existing in the art.

### BRIEF SUMMARY

The foregoing problems are addressed by the present invention. The invention comprises an introducer apparatus, and a method for percutaneously introducing a medical interventional device, such as a tracheostomy tube, into a designated area of a patient. The introducer apparatus comprises an inflatable introducer balloon that functions as a dilator/obturator. The apparatus may be used to radially dilate a tracheostomy opening with minimal longitudinal (e.g., axial) pushing forces. It also provides a smooth-transition for the distal end of the interventional device, such as a tracheostomy tube, to enable easy passage of that tube through the puncture site and avoid overdilation of the tracheal opening. Preferably, the inflatable introducer balloon has a flexible, non-traumatic distal leading end and a pre-curved balloon portion to minimize trauma to the opposite tracheal wall.

In one embodiment, the present invention comprises an introducer apparatus for use in the percutaneous placement of a medical device, such as a tracheostomy tube. The introducer apparatus comprises an inflatable balloon mounted on an elongated tube. The balloon comprises a distal portion, an intermediate portion and a proximal portion. At least a segment of the distal portion has a larger outer diameter than the outer diameter of a medical device carried on the balloon intermediate portion when the balloon is inflated. The inflated outer diameter of the intermediate portion is preferably sized relative to the internal diameter of the tracheostomy tube to securely hold the medical device thereon. The distal end portion tapers from the large diameter segment to a smaller diameter segment where the balloon meets the elongated tube. The apparatus further comprises an inflation assembly associated with the balloon for transmitting an inflation fluid to selectively inflate and deflate the balloon.

In another embodiment, the present invention comprises a method for inserting a tracheostomy tube into the trachea of a patient. The method comprises the steps of: inserting a wire guide through a tracheal opening so that a distal end of the wire guide is positioned within the trachea; providing a dilator/tracheostomy tube apparatus mounted on an elongated tube, the apparatus comprising an inflated dilator balloon having a distal portion, an intermediate portion and a proximal portion, and comprising a tracheostomy tube mounted on the intermediate portion of the dilator balloon, wherein at least a segment of the balloon distal portion has an outer diameter as least as large as the outer diameter of the tracheostomy tube, and the balloon distal end portion tapers from the large diameter segment to a smaller diameter segment where the balloon meets the elongated tube; dilating the tracheal opening by advancing the tapered distal end of the dilator/tracheostomy tube apparatus over the wire guide through the tracheal opening; continuing to advance the apparatus over the wire guide until the tracheostomy tube is positioned across the opening; deflating the dilator balloon; and withdrawing the deflated dilator balloon from the tracheal opening through a lumen of the tracheostomy tube.

In still another embodiment, the present invention comprises a method for inserting a tracheostomy tube into the trachea of a patient. The method comprises the steps of: inserting a wire guide through a tracheal opening so that a distal end of the wire guide is positioned within the trachea; providing an assembly comprising a dilator and a tracheostomy tube mounted on an elongated tube, wherein the dilator comprises an inflatable dilator balloon and the tracheostomy tube is carried by the dilator, the inflatable dilator balloon having a distal portion, and having a portion for carrying the tracheostomy tube, at least a segment of the distal portion having a generally constant diameter when the dilator balloon is inflated; inserting the dilator balloon through the tracheal opening over the wire guide such that the generally constant diameter segment spans the tracheal opening; inflating the dilator balloon such that the generally constant diameter segment expands to radially dilate the tracheal opening; advancing the dilator balloon apparatus over the wire guide until the tracheostomy tube is positioned across said opening; deflating the dilator balloon; and withdrawing the deflated dilator balloon from the tracheal opening.

### BRIEF DESCRIPTION OF THE DRAWINGS

FIG. 1 is a side elevational view of an inflatable introducer balloon apparatus according to an embodiment of the present invention;

FIG. 2 is a sectional view of the inflatable introducer balloon apparatus of FIG. 1 taken along line 2-2 of FIG. 1, with a wire guide shown passing through a lumen of the balloon;

FIG. 3 is a side elevational view of an alternative embodiment of an inflatable introducer balloon apparatus according to the present invention, utilizing a flange at the proximal end of the introducer balloon;

FIG. 4 is a side elevational view including a sectional view of the trachea, wherein an inflatable introducer balloon apparatus of the present invention having a tracheostomy tube loaded thereon is inserted into an opening of the trachea;

FIG. 5 is a sectional view of the inflatable introducer balloon and tracheostomy tube assembly of FIG. 4 taken along line 5-5 of FIG. 4, with a wire guide shown passing through a lumen of the balloon;

FIG. 6 is a side elevational view of another alternative embodiment of an inflatable introducer balloon apparatus according to the present invention having an extended distal balloon area; and

FIG. 7 is a side elevational view of another alternative embodiment of an inflatable introducer balloon apparatus according to the present invention.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

The present invention relates to an inflatable introducer balloon apparatus. In the following discussion, the terms “proximal” and “distal” will be used to describe the opposing axial ends of the apparatus, as well as the axial ends of various component features. The term “proximal” is used in its conventional sense to refer to the end of the device (or component thereof) that is closest to the operator during use of the device. The term “distal” is used in its conventional sense to refer to the end of the device (or component) that is at the greatest distance from the operator, or that is initially inserted into the patient.

FIG. 1 shows a side elevational view of one embodiment of an introducer apparatus 10 of the present invention. In this embodiment, introducer apparatus 10 comprises an inflatable introducer balloon 12 coaxially mounted on an elongated tube 14. Balloon 12 is shown in its inflated condition.

The inventive introducer apparatus is particularly useful for the percutaneous insertion of a tracheostomy tube. The apparatus is utilized to dilate a needle puncture in the trachea, thereby allowing placement of a tracheostomy tube in the dilated tracheal opening. The needle puncture and initial opening may be formed using conventional insertion techniques, such as the well-known Seldinger technique.

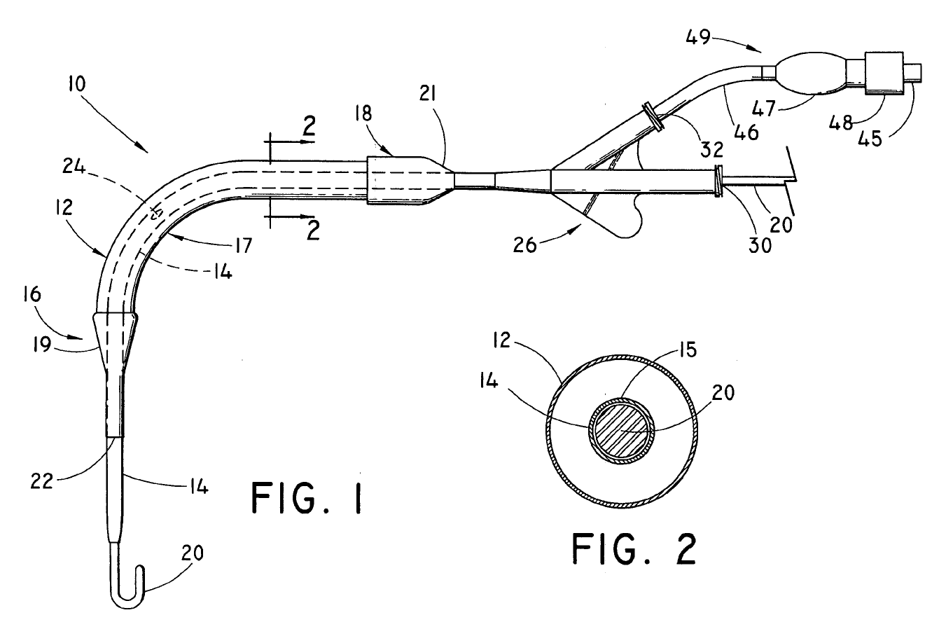

In the embodiment of FIG. 1, balloon 12 has an enlarged-diameter distal balloon portion 16 and an enlarged-diameter proximal balloon portion 18. An intermediate balloon portion 17 is situated between distal balloon portion 16 and proximal balloon portion 18. In the embodiment shown, intermediate portion 17 has a smaller outer diameter than distal and proximal balloon portions 16, 18. Tube 14 has a central lumen 15 extending therethrough. Lumen 15 is sized to permit passage therethrough of a wire guide 20 (FIG. 2).

A conventional proximal fitting 26 is bonded or otherwise attached to the proximal end of tube 14 in well-known fashion. A tapered end 21 of enlarged diameter proximal portion 18 is attached to tube 14 by any well-known means, such as heat bonding or adhesion, to seal the proximal end of balloon 12. Alternatively, the proximal end of balloon 12 can be closed off by bonding or otherwise attaching it directly to proximal fitting 26. Similarly, a tapered end 19 of enlarged diameter distal portion 16 is bonded to tube 14 by any well-known means to close off the distal end of balloon 12.

Proximal fittings such as fitting 26 normally include a plurality of extensions, or ports, for attachment to other devices, or to define a passageway through the fitting. In the embodiment shown, fitting 26 includes two ports, namely, a wire guide port 30 and an inflation port 32. Port 30 is sized for passage of wire guide 20. Inflation port 32 communicates with one end of an inflation tube 46. The other end of inflation tube 46 communicates with a conventional inflation assembly 49. In the embodiment shown, inflation assembly 49 comprises a pilot balloon 47 and a one-way valve 48. Those skilled in the art will appreciate that any conventional inflation assembly may be substituted for assembly 49. An inflation fluid, such as air, is received from a fluid source (not shown) through end 45 of the inflation assembly 49.

During inflation of balloon 12, the inflation fluid from the fluid source passes through inflation assembly 49 and inflation tube 46, and thereafter through central lumen 15 of tube 14. The fluid then inflates balloon 12 by passing from lumen 15 into the interior space of the balloon through one or more openings 24 in tube 14. Alternatively, instead of transmitting the inflation fluid through central lumen 15, a second lumen can be provided for transmitting inflation fluid in well-known manner from a fluid source to the interior of the balloon. The second lumen can be coaxial with lumen 15, or be situated adjacent lumen 15. As still another alternative, the second lumen can be provided in a second tube positioned generally adjacent tube 14.

The distal end portion 16 of balloon 12 tapers from an enlarged area to a tapered end portion 19. The distal portion of balloon 12 may be provided with a gradual taper in the distal direction to a point 22 where the balloon meets and is bonded or otherwise adhered to the outer surface of tube 14. If desired, tube 14 can extend well beyond meeting point 22 to provide a smaller diameter, flexible, non-traumatic leading end to the system. This tapering and flexibility inhibits trauma to the opposite tracheal wall as the system is inserted and advanced into place. This flexible leading end portion preferably ranges from 2 to 10 cm in length.

In the embodiment of FIG. 1, enlarged diameter proximal portion 18 of balloon 12 has an outside diameter of sufficient size when the balloon is inflated to block or otherwise obstruct the axial movement in the proximal direction of a tracheostomy tube carried on the intermediate portion of the introducer apparatus, as the introducer apparatus is advanced through the puncture site and into the trachea.

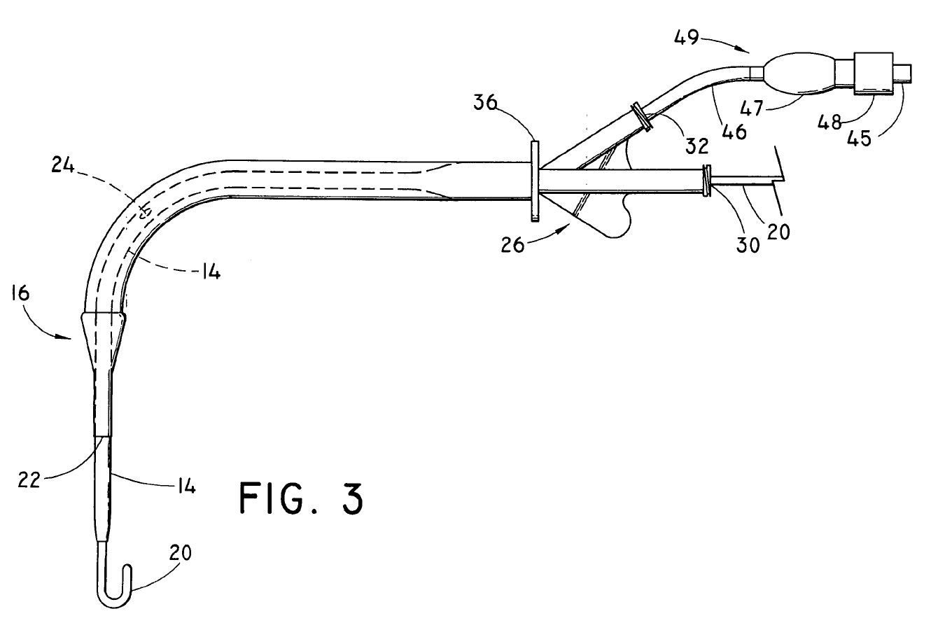

An alternative embodiment of an inflatable introducer balloon assembly is shown in FIG. 3. In this embodiment, the enlarged proximal portion of balloon 12 shown in the previous embodiment has been omitted, and replaced with a flange 36 or similar structure to obstruct proximal movement of the tracheostomy tube. Flange 36 is mounted at or near the proximal end of balloon 12. Alternatively, flange 36 can be provided as a part of the proximal fitting 26. The flange or alternative fitting maintains the tracheostomy tube in position on the intermediate portion of the balloon by obstructing axial movement of the tracheostomy tube relative to the introducer apparatus as the assembly is introduced into the tracheal opening. Providing a flange 36 instead of an enlarged proximal portion 18 may simplify manufacture of balloon 12.

In a preferred embodiment, the outer diameter of the intermediate portion 17 of the balloon 12 has a diameter when fully inflated that is equal to or slightly larger than the inside diameter of the tracheostomy tube that is carried by the intermediate portion. In this manner, the tracheostomy tube is mounted on and securely held by the intermediate portion 17 of balloon 12, as the balloon introducer/tracheostomy tube assembly is advanced into the trachea through the puncture site.

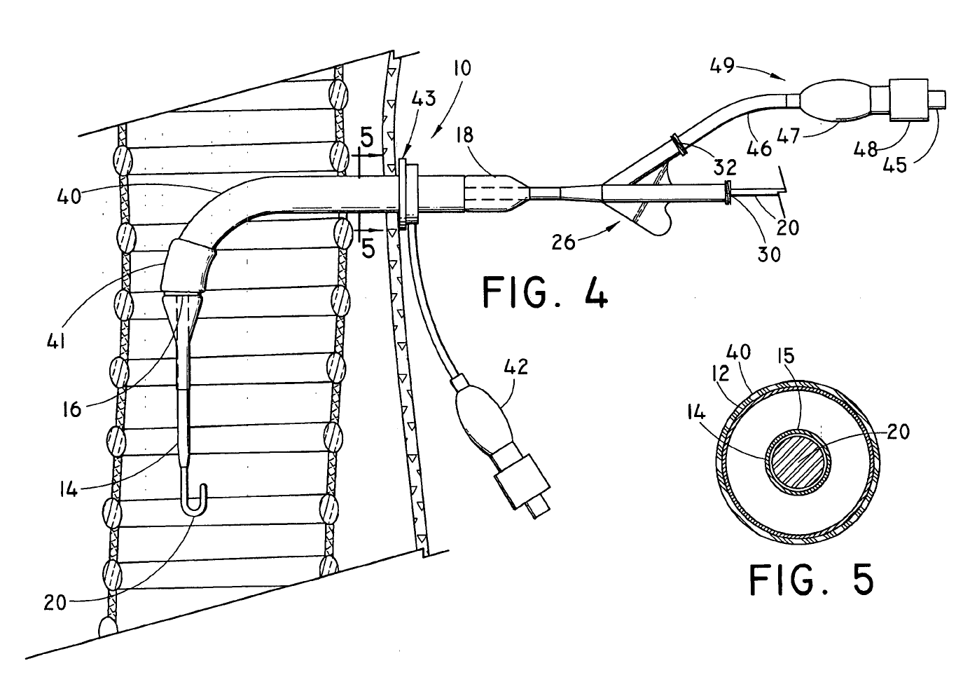

FIGS. 4 and 5 illustrate an arrangement wherein tracheostomy tube 40 is mounted on the intermediate portion 17 of the balloon introducer. In FIG. 4, the assembly is shown in position in the trachea of a patient. In this figure, tracheostomy tube 40 is also provided with an optional inflatable cuff 41, flange 43, and inflation assembly 49. The optional cuff 41, flange 43, and inflation assembly 49 are well known accessories that are commonly used with tracheostomy tubes.

Enlarged distal balloon portion 16 preferably has a maximum outer diameter when fully inflated that is equal to or slightly larger than the outer diameter of the tracheostomy tube. Upon insertion of the introducer balloon into a tracheal opening, this large diameter balloon portion dilates the puncture site sufficiently to provide for a smooth introduction of the tracheostomy tube through the puncture site and into the trachea.

Prior art dilators are not provided with an enlarged distal end to provide a smooth transition from the proximal end of the inserted dilator to the distal end of the tracheostomy tube. As a result, insertion of such an assembly frequently causes a “bump” after the smaller diameter dilator has passed through the tracheal opening, and the opening thereafter encounters the larger diameter distal end of the tracheostomy tube. The bump occurs as the larger diameter tracheostomy tube is forced through the smaller diameter opening formed by the dilator.

Since an insertion dilator of the type known in the art must be withdrawn through the interior of the tracheostomy tube following placement of the tube, a prior art dilator could not be physically withdrawn from the tracheostomy tube if its outer diameter was larger than the inner diameter of the tracheostomy tube through which it must be withdrawn. The inventive introducer (dilator) comprises an inflatable balloon. As a result, the balloon can be deflated following insertion and placement of the tracheostomy tube, and can thereafter be easily withdrawn through the tracheostomy tube following proper placement of the tube. Since it is no longer necessary to initially insert a smaller diameter dilator through a tracheal opening, to be followed by the insertion of a larger diameter tracheostomy tube, insertion of the tracheostomy tube is a smoother operation, as the bump has been eliminated.

The distal extension portion of tube 14 and the distal balloon tapered portion are preferably coated with a suitable lubricant such as a conventional hydrophilic coating. Such coatings are well known to those skilled in the art, and are commonly used on vascular wire guides and dilators to ease the introduction of the device through a body opening. One such hydrophilic coating is SLIP COAT, available from STS, New York. Other biocompatible lubricants, such as silicone, can also be utilized to substantially reduce the amount of longitudinal (axial) force or push needed to insert the system into the trachea.

Tube 14 can be made of a conventional semi-rigid polymer commonly used in the medical arts. Preferably tube 14 will be a thermoplastic polymer, such as nylon, polyethylene, polyurethane or PVC.

Balloon portion 12 can be made of any well-known material commonly used for balloons in medical applications, such as balloons used for dilating vascular stenoses. These materials include, among others, PET, cross-linked Nylon, polyethylene, PVC and fiber reinforced elastomers. The balloon can be blow molded by conventional methods in a mold formed to the desired shape and curvature of the balloon. Preferably, the balloon is blow molded to include the desired curvature, although the balloon can be formed and used in a straight configuration if desired. Alternatively, the balloon can be made by spraying or dip coating a mold or forming mandrel with a plastisol or thermoplastic elastomer dissolved in a solvent. As still another alternative, the balloon can be formed to incorporate other known materials such as a reinforcing braid, spiral wrap, fibers, etc. Such materials may be utilized, for example, to provide additional strength and reinforcement to the balloon to enable it to withstand any sharp fragments and the like that the balloon may come in contact with as it passes into the trachea.

Use of the inflatable introducer balloon assembly 10 in connection with the introduction of a tracheostomy tube will now be described. Initially, the physician makes an entry into the trachea, such as by inserting a needle through the tracheal tissue and cartilage. Preferably, a needle of about 18 gage (0.052 in; 1.32 mm O.D.) is utilized to make the initial puncture, although those skilled in the art will recognize that a larger, or smaller, gage needle may be utilized in a particular situation. A wire guide is inserted through the needle and into the trachea in well-known fashion. The wire guide can be any conventional wire guide commonly used for such purposes, such as a floppy tip, “J” type TSCFB-38-60-3.0 wire, available from Cook, Inc., Bloomington, Ind., shown in the drawings. Following introduction of the wire guide, the needle is then withdrawn, leaving the wire guide in place.

Tapered distal end portion 19 of an inflated introducer balloon 12 is then advanced directly over the wire guide, through the puncture site and into the trachea. Preferably, introducer balloon 12 is provided with tracheostomy tube 40 securely mounted at intermediate portion 17 of the inflated balloon, as shown in FIG. 4. As the introducer balloon assembly is advanced into the trachea, the tracheal opening is dilated until the enlarged distal balloon portion 16 passes fully through the puncture site. The introducer balloon is further inserted until the physician determines that tracheostomy tube 40 has been properly positioned across the tracheal wall. The introducer balloon is then deflated in any conventional fashion, such as by evacuating the inflation fluid back through port 32. Preferably, port 32 is provided with a conventional valve system to enable the physician to maintain the balloon in the inflated or pressurized condition until the physician determines that it is time to release the pressure or deflate the balloon for removal of the balloon introducer. The deflated balloon is then removed through the center lumen of the tracheostomy tube. Once it has been properly positioned, the tracheostomy tube may be anchored to the patient in conventional fashion, such as by a strap around the patient's neck.

In the preferred embodiment, the introducer balloon assembly 10 is pre-curved as shown to allow the system to easily turn and travel down the trachea as it is advanced. In a still further preferred embodiment, the curvature of the introducer balloon assembly corresponds with the curvature of the tracheostomy tube.

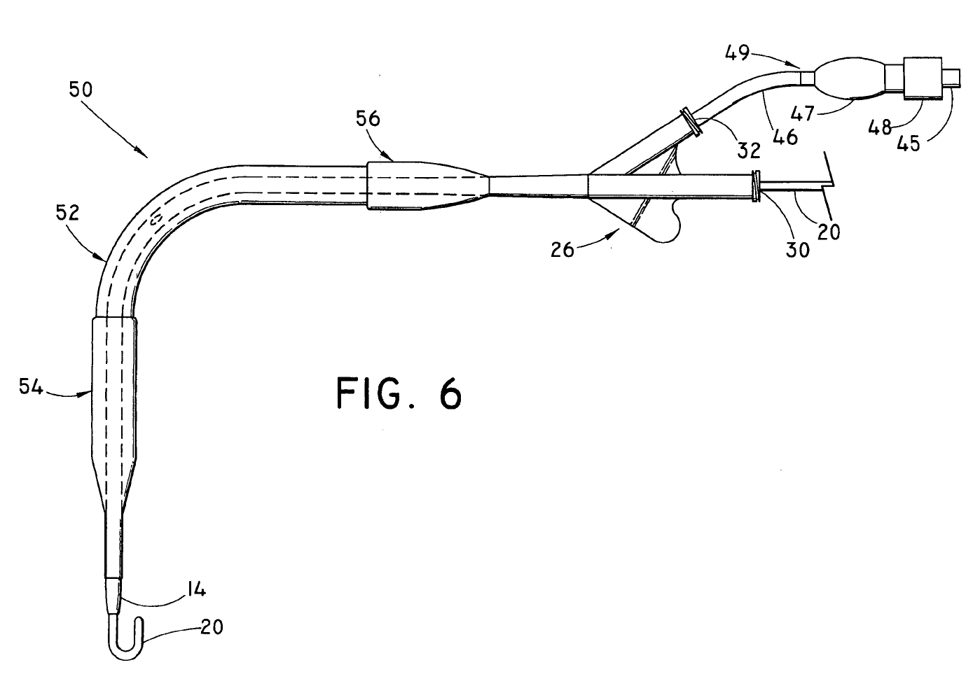

Another alternative embodiment of an introducer balloon assembly 50 is illustrated in FIG. 6. In this embodiment, the introducer balloon 52 includes an enlarged proximal portion 56, and a generally constant diameter distal portion 54 that has an extended length when compared to the length of the distal portion of the prior embodiments. With this embodiment, the introducer balloon may be initially inserted in the deflated condition, or in a partially inflated, low profile condition. Distal balloon portion 54 is passed through the puncture site to a point wherein some (but not all) of extended distal portion 54 has passed through the puncture site, and the tracheostomy tube (not shown) has not yet reached the puncture site. The introducer balloon 52 is then inflated to its full size and force to radially dilate the opening. With this embodiment, most, if not all, of the axial, or push, force that would otherwise be exerted against the tracheal wall during axial insertion of the dilator has been eliminated. When inflated, the outer diameter of extended distal portion 54 is similar in size to the maximum outer diameter of enlarged portion 16 of the embodiment of FIG. 1.

The extended distal balloon embodiment of FIG. 6 lends itself well to this procedure since it has a longer length of maximum diameter balloon ahead of (i.e., distal to) the tracheostomy tube, thereby allowing the dilator to engage more tissue before the leading end of the tracheostomy tube enters the puncture site. This embodiment is expected to be particularly useful for grossly overweight or obese patients.

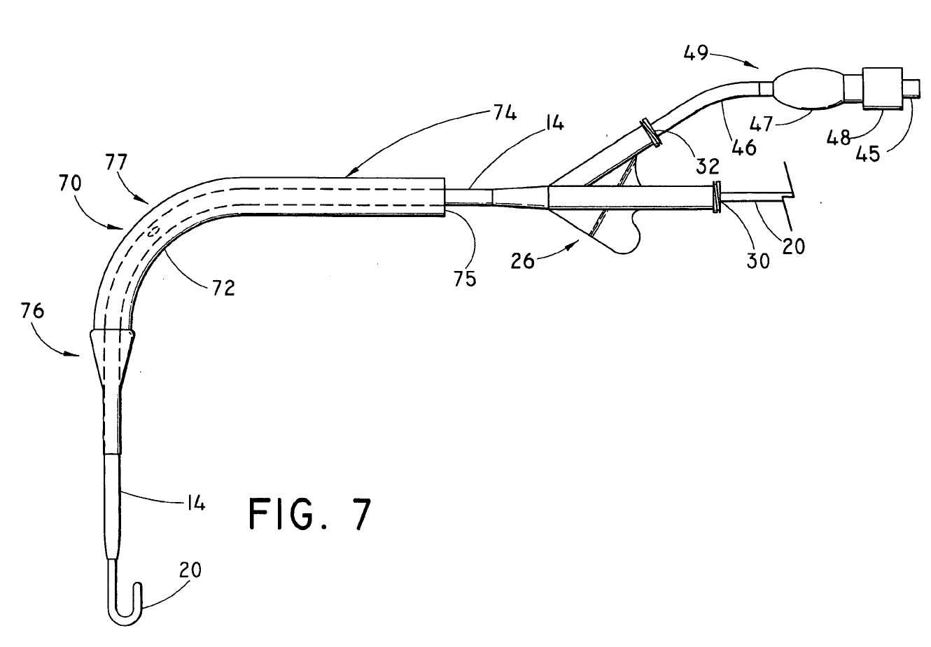

Yet another alternative embodiment of an introducer balloon assembly 70 is illustrated in FIG. 7. In this embodiment, balloon 72 may be configured with an enlarged distal portion 76 in a manner generally similar to the balloons described in the previous embodiments. However, in this embodiment the presence of an enlargement at the proximal end, such as enlarged balloon portion 18 in the embodiment of FIG. 1 or the flange 36 in the embodiment of FIG. 3, has been eliminated. Proximal end 74 of balloon 72 may include an abrupt terminal end 75 as shown in the figure, or the terminal end may be tapered to tube 14 as previously described. Since the outer diameter of the intermediate portion 77 of the inflated balloon has a diameter that is equal to or slightly larger than the inside diameter of the tracheostomy tube loaded thereon, the engagement between the inflated balloon and the tracheostomy tube will generally be sufficiently secure such that a proximal enlargement is not necessary.

The inventive introducer balloon of the present invention has many advantages when compared to dilators/obturators of the prior art. Such prior art devices do not have an enlarged distal end to provide a smooth transition to the leading end of the tracheostomy tube as described. If such prior art devices were provided with an enlarged distal end, the distal end could not be reduced in diameter to allow for withdrawal of the dilator following placement of the tracheostomy tube. The introducer balloon of the present invention is not limited in this manner since it can be simply deflated to a smaller diameter and easily removed through the interior of the tracheostomy tube. In addition, insertion of the dilator can be less traumatic to the patient than insertion of prior art dilators, since the axial, or push, force against the tracheal wall has been minimized.

The inventive introducer balloon assembly is also useful for removing a tracheostomy tube. Since the introducer balloon, when inflated, locks or grips the inside surface of the tracheostomy tube along the full length of the tube, the physician has a much better grip on the tracheostomy tube for manipulation and removal than was previously available. If a tracheostomy tube is to be removed and replaced with another tube, a wire guide can be introduced through the balloon dilator/obturator and the dilator can be positioned and inflated such that it securely grips the old tracheostomy tube. The dilator and tracheostomy tube can then be withdrawn. The wire can then be left behind to aid the insertion of the new tracheostomy tube.

The introducer balloon apparatus can be supplied separately to the physician, or alternatively, it can be supplied in combination with the tracheostomy tube. With the combination, the tracheostomy tube can be pre-mounted on the introducer balloon. In this case, the assembly includes the introducer balloon as well as the tracheostomy tube. In addition, the introducer balloon, and/or the balloon/tracheostomy tube combination can be provided as part of a kit that contains some or all of the miscellaneous ancillary products that are useful in the procedure. Among the ancillary products that can be packaged together in a package if desired are a needle, a wire guide, and an anesthetic (such as lidocaine). If desired, the package can also be designed to serve as a tray for holding and organizing the components.

Although the inventive dilator apparatus has been primarily described for use in connection with a tracheostomy tube, the invention is not so limited. Rather, those skilled in the art will appreciate that it can be used in other percutaneous entry techniques whenever a medical device, such as a shaft or sheath, is to be positioned at a designated area of a patient.

It is therefore intended that the foregoing detailed description be regarded as illustrative rather than limiting, and that it be understood that it is the following claims, including all equivalents, that are intended to define the spirit and scope of this invention.

1. An introducer apparatus for use in the percutaneous placement of a medical device, comprising:
an elongated tube, said elongated tube having a lumen extending therethrough;
an inflatable balloon mounted on said elongated tube, the balloon comprising a distal portion, an intermediate portion and a proximal portion, at least a segment of the distal portion having an inflated outer diameter as least as large as an outer diameter of a medical device carried by the intermediate portion of said balloon; and
an inflation assembly associated with the balloon for transmitting an inflation fluid to inflate the balloon.

2. The introducer apparatus of claim 1, wherein said medical device comprises a tracheostomy tube, said intermediate portion having an inflated outer diameter at least as large as an inner diameter of said tracheostomy tube such that said tracheostomy tube is mounted on said intermediate portion, and wherein said balloon distal end portion tapers from said large diameter segment to a smaller diameter segment where said balloon meets said elongated tube.

3. The introducer apparatus of claim 1, wherein the proximal portion of said inflatable balloon has a larger outer diameter than the outer diameter of the intermediate portion when said balloon is inflated.

4. The introducer apparatus of claim 1, further comprising a flange at said proximal portion of said balloon, said flange having a larger diameter than the outer diameter of the medical device.

5. The introducer apparatus of claim 1, wherein said distal portion of said inflatable balloon includes a segment of substantially constant diameter when inflated, said substantially constant diameter being larger than the diameter of the intermediate portion when said balloon is inflated.

6. The introducer apparatus of claim 2, wherein said diameter of said large diameter segment of said distal portion is larger than the outer diameter of said tracheostomy tube.

7. The introducer apparatus of claim 1, wherein said lumen of said elongated tube includes a radial opening for permitting said inflation fluid to traverse through at least a portion of said lumen to an interior space of said balloon, said inflation assembly further being operable for selectively inflating and deflating said balloon.

8. The introducer apparatus of claim 1, further comprising a fitting at a proximal end of said elongated tube.

9. The introducer apparatus of claim 8, wherein said fitting includes a port for passage of a wire guide and a port for passage of an inflation fluid.

10. The introducer apparatus of claim 1, wherein said balloon comprises at least one of PET, cross-linked Nylon, polyethylene, PVC and a fiber reinforced elastomer.

11. The introducer apparatus of claim 1, wherein said elongated tube comprises a thermoplastic polymer.

12. The introducer apparatus of claim 1, wherein said balloon has a curved orientation when inflated.

13. The introducer apparatus of claim 1, wherein said segment of said distal portion has a larger outer diameter that the outer diameter of the intermediate portion when said balloon is inflated.

14. The introducer apparatus of claim 2, wherein said balloon has a curved orientation when inflated, said curved orientation corresponding to a bend in said tracheostomy tube.

15. A method for inserting a tracheostomy tube into the trachea of a patient, comprising:
inserting a wire guide through a tracheal opening so that a distal end of the wire guide is positioned within the trachea;
providing a dilator/tracheostomy tube apparatus mounted on an elongated tube, the apparatus comprising an inflated dilator balloon having a distal portion, an intermediate portion and a proximal portion, and comprising a tracheostomy tube carried on said intermediate portion of the dilator balloon, at least a segment of the balloon distal portion having an outer diameter as least as large as the outer diameter of the tracheostomy tube, said balloon distal end portion tapering from said large diameter segment to a smaller diameter segment where said balloon meets said elongated tube;
dilating said tracheal opening by advancing the tapered distal end of the dilator/tracheostomy tube apparatus over said wire guide through said tracheal opening;
continuing to advance the apparatus over said wire guide until the tracheostomy tube is positioned across said opening;
deflating said dilator balloon; and
withdrawing the deflated dilator balloon from the tracheal opening through a lumen of the tracheostomy tube.

16. The method of claim 15, wherein said wire guide is inserted into said tracheal opening by inserting a needle into said tracheal wall, and inserting said wire guide into said tracheal opening through a bore of said needle, said method further comprising the step of withdrawing said needle, leaving said wire guide in place.

17. The method of claim 15, wherein said balloon comprises at least one of PET, cross-linked Nylon, polyethylene, PVC and a fiber reinforced elastomer.

18. The method of claim 15, wherein said inflated diameter of said large diameter segment of said distal portion is larger than the outer diameter of said tracheostomy tube.

19. A method for inserting a tracheostomy tube into the trachea of a patient, comprising:
inserting a wire guide through a tracheal opening so that a distal end of the wire guide is positioned within the trachea;
providing an assembly comprising a dilator and a tracheostomy tube mounted on an elongated tube, said dilator comprising an inflatable dilator balloon and said tracheostomy tube carried by said dilator, the inflatable dilator balloon having a distal portion, and having a portion for carrying said tracheostomy tube, at least a segment of said distal portion having a generally constant diameter when said dilator balloon is inflated;
inserting said dilator balloon through said tracheal opening over said wire guide such that said generally constant diameter segment spans said tracheal opening;
inflating said dilator balloon such that said generally constant diameter segment expands to radially dilate said tracheal opening;
advancing the dilator balloon apparatus over said wire guide until the tracheostomy tube is positioned across said opening;
deflating said dilator balloon; and
withdrawing the deflated dilator balloon from the tracheal opening.

20. The method of claim 19, wherein said constant diameter portion has an outer diameter at least as large as an outer diameter of said tracheostomy tube.
